(19) United States
(12) Patent Application Publication
(10) Pub. No.: US 2005/0089486 A1
(43) Pub. Date: Apr. 28, 2005

(54) Self-tanning compositions and method of using the same

(76) Inventors: Ralph Spindler, Palatine, IL (US); Ray Vakili, Naperville, IL (US); Stephen J. Urbanec, Arlington Heights, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/948,657

(22) Filed: Sep. 23, 2004

Related U.S. Application Data
(60) Provisional application No. 60/505,660, filed on Sep. 24, 2003.

(51) Int. Cl. A61K 7/42; A61K 31/715

(52) U.S. Cl. 424/59; 514/61

(57) ABSTRACT

A composition and method of enhancing the tanning rate of a self-tanning composition is disclosed. The self-tanning composition contains an amine potentiator loaded onto a microparticle delivery system.

### CROSS REFERENCE TO RELATED APPLICATION

This application claims the benefit of provisional U.S. provisional Ser. No. 60/505,660, filed Sep. 24, 2003.

### FIELD OF THE INVENTION

The present invention is directed to a composition and a method of enhancing the rate of tanning of self-tanning compositions with a minimal effect on the color of the composition. More particularly, the present invention is directed to a self-tanning composition containing an amine potentiator loaded on a microparticle delivery system.

### BACKGROUND OF THE INVENTION

The darkening of light colored skin, either through exposure to ultraviolet radiation or through the use of chemical-based tanning compositions, is highly desirable to a relatively large portion of the population. Many individuals avoid unnecessary exposure to ultraviolet radiation due to the increased risk of skin cancer after extensive exposure. Therefore, alternative means of darkening the skin have increased in popularity.

One of the most widely used methods of enhancing skin color is by the application of dihydroxyacetone (DHA), in a suitable cosmetic formulation, to the skin. DHA forms a dimeric structure that degrades to monomeric DHA. Monomeric DHA darkens the skin through a reaction similar to the Maillard reaction by reacting with free amino groups of skin proteins. Initially, the skin color formed after an application of DHA was unpredictable, and often was an orange hue rather than the desired brown color. Through the use of more highly purified DHA, and improved formulations containing DHA, self-tanning formulations now are more effective in producing the desired brown skin color.

One significant disadvantage of the DHA self-tanning approach is the length of time (e.g., more than 6 to 12 hours) required to observe a demonstrable darkening of the skin. Several different approaches have been used in an attempt to improve the speed of the tanning process, including adding compounds, known as potentiators, to self-tanning formulations. Typically, potentiators are primary or secondary amino-containing compounds, such that the DHA will react with skin proteins to produce a brown color. With proper formulation of a potentiator, it also is possible to provide a more natural tan color.

Although potentiators help shorten the time wherein the results of self-tanning are observed, the self-tanning formulations containing a potentiator often are unstable with respect to color formation in the container. From a consumer acceptance standpoint, this is a serious esthetic disadvantage, and, furthermore, DHA that is reacting with the potentiator no longer is available to tan the skin and, therefore, the tanning effectiveness of the formulation is reduced. Several methods to overcome the problem of unwanted color formation have been proposed, including first applying a potentiator solution to the skin, followed by an application of a DHA-containing formulation, or a vice versa application with a first application of DHA, then the potentiator (see, U.S. Pat. Nos. 5,503,874; 5,705,145; 5,705,145; and 6,399,048). Another approach utilizes a two-chamber package wherein one chamber contains an emulsion incorporating a potentiator and the second chamber contains an emulsion incorporating DHA (see, U.S. Pat. Nos. 5,645,822 and 5,750,092). Upon application to the skin, the contents of the two chambers mix such that the potentiator can activate the DHA to enhance the rate of tanning. This approach is highly effective, but the cost of developing dual chamber packaging, and the cost to consumers, can be prohibitively expensive.

The present invention is directed to over-coming disadvantages associated with prior self-tanning compositions by loading a potentiator compound into a delivery system, including the loaded delivery system in a self-tanning composition that contains DHA or other self-tanning compound, thereby preventing the potentiator from prematurely reacting with the DHA until the formulation is applied to the skin. Premature darkening of the self-tanning composition therefore is avoided.

### SUMMARY OF THE INVENTION

The present invention is directed to self-tanning compositions. More particularly, the present invention is directed to a composition containing both a self-tanning compound and a potentiator for the self-tanning compound.

Therefore, one aspect of the present invention is to provide a composition comprising a self-tanning compound and a potentiator, wherein the potentiator is loaded onto polymeric microparticles.

Another aspect of the present invention is to provide a method of self-tanning comprising applying a single composition to the skin, wherein the time to achieve a desired tan color is reduced.

Still another aspect of the present invention is to provide a color-stable self-tanning composition comprising a self-tanning compound and a potentiator loaded onto polymeric microspheres.

These and other novel aspects of the present invention will become apparent from the following detailed description of the preferred embodiments.

### DETAILED DISCUSSION OF THE PREFERRED EMBODIMENTS

Delivery systems routinely are used in personal care and pharmaceutical topical compositions to extend the useful life of an active compositions pound, to protect the active compound from decomposition in the composition, or to enable or facilitate formulation of the active compound into a composition due to problems such as solubility or aesthetics. A delivery system that can provide all these advantages is the adsorbent microparticle. One preferred class of adsorbent microparticle polymers useful as a delivery system is prepared by a suspension polymerization technique, as set forth in U.S. Pat. Nos. 5,677,407; 5,712,358; 5,777,054; 5,830,967; and 5,834,577, each incorporated herein by reference. Such an adsorbent polymer is sold under the tradename of POLY-PORE® E200, available from AMCOL International Corporation, Arlington Heights, Ill.

Another preferred class of adsorbent microparticle polymers useful as a delivery system is prepared by a precipitation polymerization technique, as set forth in U.S. Pat. Nos. 5,830,960 and 5,837,790, both incorporated herein by reference. Such an adsorbent polymer is sold under the tradename POLY-PORE® L200, also available from AMCOL International Corp.

These adsorbent microparticle polymers also can be modified after the incorporation of an active compound to modify the rate of release of such a compound as set forth in U.S. Pat. No. 6,491,953, incorporated herein by reference.

Another adsorbent polymer that is prepared by a precipitation polymerization technique is set forth in U.S. Pat. Nos. 4,962,170; 4,948,818; and 4,962,133, is sold under the tradename of POLYTRAP®, also available from AMCOL International Corp. Other adsorbent polymers are commercially available include, for example, MICROSPONGE® (a copolymer of methyl methacrylate and ethylene glycol dimethacrylate), available from Cardinal Health, Sommerset, N.J., and Poly-HIPE polymers (e.g., a copolymer of 2-ethylhexyl acrylate, styrene, and divinylbenzene) available from Biopore Corporation, Mountain View, Calif.

To provide a delivery system for an active compound, the active compound, e.g., a potentiator is incorporated, or loaded, onto, or into, the microparticles. This is accomplished by spraying or adding the compound directly to the microparticle delivery system in a manner such that a homogeneous distribution of the active compound on the microparticles is achieved. As used herein, the active compound is “loaded” onto the delivery system, i.e., is adsorbed, absorbed, and/or entrapped in the microparticle delivery system.

Alternatively, the active compound first can be dissolved in a suitable solvent, then the resulting solution is sprayed or added to the microparticle delivery system. The solvent then is removed by heating, vacuum, or both. As previously stated, two or more different types of materials can be added to the microparticle, wherein one of the materials is an active compound and the other material is used either to modify the release rate of the active compound from the microparticles, and/or to protect the active compound loaded in the microparticles from reacting or otherwise interacting with other ingredients contained in the final formulation. These release modifying or protective materials can be added in their molten state directly to the microparticles or first dissolved in a suitable solvent, sprayed onto the microparticles, and followed by removal of the solvent from the delivery system.

Potentiators that can be used to increase the rate of self-tanning, or the deepness of the tan, generally include amino-containing compounds. Self-tanning potentiators include amino acids, like lysine, arginine, and glycine, and compounds that contain amino groups, like diamines, triamines, and higher order amines such as 1,2-ethanediamine, 1,3-propanediamine, 1,4-butanediamine, 1,6-hexamethylenediamine, diethylenetriamine, triethylenetetra-amine, or derivatives or isomers of these amine compounds.

Other useful amine potentiators include, but are not limited to, N,N′-dimethylethylenediamine, N,N′-diethylethylenediamine, N,N′-diisopropylethylenediamine, N,N′-di-n-propylethylenediamine, N,N′-di-n-butylethylenediamine, N,N′-di-n-hexylethylenediamine, N,N′-dibenzylethylenediamine, N,N′-di-(2-carboxyethyl)-ethylenediamine, N,N′-di-(2-hydroxyethyl)ethylenediamine, N-ethylethylenediamine, N-n-propylethylenediamine, N-isopropyl-ethylenediamine, N-n-butylethylenediamine, N-sec-butylethylenediamine, N-hexylethylenediamine, N-phenylethylenediamine, N-benzylethylenediamine, N-(2-hydroxyethyl)-ethylenediamine, N-(3-hydroxy-propyl)-ethylenediamine, N-[3-(trihydroxysilyl)-propyl]-ethylenediamine, N-[3-(trimethoxysilyl)-propyl]-ethylenediamine, and N-naphthylethylenediamine. Other diamine and derivatives of diamines are disclosed in U.S. Pat. Nos. 5,750,092 and 5,645,822, each incorporated herein by reference.

Polymeric amino-containing compounds useful as potentiators include, but are not limited to, siloxane polymers having pendant amino groups, such as those available from General Electric, Schenectady, N.Y. (e.g., GE SF 1706 or GE SF 1708) or Dow Corning Corp., Midland, Mich.(e.g., DC 2-8566). Each of these amino-modified silicone polymers is known by the designated INCI name of amodimethicone. Methoxy amodimethicone/silesquioxane copolymer also can be used as a potentiator. Linear polyethylene-diamine, or branched versions of a similar polymer, also can be used as a potentiator, as can polyethylenimines, dendritic versions of amino polymers, such as those available from by Dendritech, Inc., Midland, Mich., (PAMAM dendrimers) or from DSM, Galeen, Netherlands. Polyethyleneimines of the formula (CH₂CH₂NH)ₙ wherein n ranges 30 to 15,000, such as the EPOMIN™ products available from Aceto Corporation, Flushing, N.Y., U.S.A., and the POLYMIN™ products are available from BASF Corporation, Parsippany, N.J., U.S.A. In addition, polymeric versions of amino acids, such as poly(lysine) and poly(argine), can be used as a potentiator.

In another embodiment, the amino-containing potentiator first is loaded onto a microparticle delivery system, followed by the addition of second material which modifies the rate of release of the potentiator when the self-tanning composition has been applied to the skin or protects the potentiator loaded on the microparticle from prematurely reacting with other compounds contained in the formulation, like DHA.

Examples of such a modifying compound are low melting (C₈ through C₂₀)alcohols and fatty alcohols ethoxylated with one to three moles of ethylene oxide. Examples of fatty alcohols and ethoxylated fatty alcohols include, but are not limited to, behenyl alcohol, caprylic alcohol, cetyl alcohol, cetaryl alcohol, decyl alcohol, lauryl alcohol, isocetyl alcohol, myristyl alcohol, oleyl alcohol, stearyl alcohol, tallow alcohol, steareth-2, ceteth-1, cetearth-3, and laureth-2. Additional fatty alcohols and ethoxylated alcohols are listed in the “International Cosmetic Ingredient Dictionary and Handbook, Tenth Edition, Volume 3” (2004), pages 2127 and pages 2067-2073, incorporated herein by reference. Another class of modifying compounds are the C₈ to C₂₀ fatty acids, including, but not limited to, stearic acid, capric acid, behenic acid, caprylic acid, lauric acid, myristic acid, tallow acid, oleic acid, palmitic acid, isostearic acid, and additional fatty acids listed in the “International Cosmetic Ingredient Dictionary and Handbook, Tenth Edition, Volume 3,” page 2126-2127, incorporated herein by reference.

The modifying compound also can be hydrocarbon, like mineral oil, 1-decene dimer, polydecene, paraffin, petrolatum, vegetable-derived petrolatum, or isoparaffin. Another class of modifying compounds is waxes, like mink wax, carnauba wax, and candelilla wax, for example, and synthetic waxes, like silicone waxes, polyethylene, and polypropylene. Fats and oils also can be useful modifying compounds which include, for example, but are not limited to, lanolin oil, linseed oil, coconut oil, olive oil, menhaden oil, castor oil, soybean oil, tall oil, rapeseed oil, palm oil, and neatsfoot oil, and additional fats and oils listed in the “International Cosmetic Ingredient Dictionary and Handbook, Tenth Edition, Volume 3,” pages 2124-2126. Other useful modifying compounds are water-insoluble esters having at least 10 carbon atoms, and preferably 10 to about 32 carbon atoms. Numerous esters are listed in “International Cosmetic Ingredient Dictionary and Handbook, Tenth Edition,” pages 2115-2123.

The potentiator also can be mixed with a suitable nonreactive diluent, before the addition of the modifying protectant or release material, if desired. Such diluents include, but are not limited to, silicone fluids, like cyclomethicone or dimethicone; ester solvents, like caprylic/capric triglyceride; hydrocarbons, like isododecane; and other appropriate diluents and mixtures thereof.

Self-tanning compositions of the present invention can be prepared in a variety of formulation types, including, for example, oil in water emulsions (o/w), water in oil emulsions (w/o), anhydrous sticks, and aqueous gels. A microparticle delivery system of the present invention can be incorporated into any of these formulation types. For example, an o/w-emulsion can be prepared, then microparticles loaded with a potentiator can be added to the emulsion, preferably when preservatives or fragrances are being added to the emulsion. Sufficient agitation is supplied to the emulsion to ensure that the microparticle delivery system is homogeneously mixed into the composition. A similar protocol can be used to prepare other product types.

A self-tanning composition of the present invention contains a self-tanning compound in a sufficient amount to achieve a desired degree of tanning. The amount of self-tanning compound in the composition is well known to persons skilled in the art, but typically is about 0.1% to about 10%, preferably about 1% to about 7.5%, and more preferably about 1% to about 5%, by weight.

The amount of potentiator included in the composition is sufficient to enhance the rate of tanning over a composition containing the same self-tanning composition, in the same amount, but absent a potentiator. Typically, a potentiator is present in the self-tanning composition in an amount of about 0.1% to about 10%, preferably about 1% to about 5%, and most preferably about 0.1% to about 2%, by weight.

The potentiator is incorporated into the self-tanning composition after loading onto polymeric microparticles. The amount of microparticles in the composition is related to the desired amount of potentiator in the composition, and the amount of potentiator loaded onto the microparticles. Typically the potentiator is loaded onto a polymeric microspheres in an amount such that the loaded microspheres contain about 2% to about 80%, preferably about 5% to about 70%, and more preferably about 5% to about 50%, by weight, of the potentiator.

The release mechanism of the potentiator from the microparticles onto the skin either can be from diffusion of the potentiator out of the microparticle delivery system or a release of the potentiator through physical attrition of the microparticle by the action of applying the composition to the skin. These mechanisms allow the potentiator to essentially form a film on the skin that can then react with the DHA, L-erythrulose, or other self-tanning compound in the composition.

To determine the rate of tan development, an in vitro technique described by R. Jermann et al., International Journal of Cosmetic Chemistry (2002), 24, 1-8). In this method, VITRO-SKIN™ (IMS, Milford, Conn.) was used as a substrate because it is similar to human skin in that it reacts with DHA to form a brown color. Color development can be tracked as a function of time by using a color meter (e.g., a Labscan 2 from Hunter Lab). The color meter measures the L*, a*, and b* color parameters which can be compared the same values for the original VITRO-SKIN™ substrate using the following equation: ΔE=((L*(0)-L*(t))²+(a*(0)-a*(t) )²+(b*(0)-b*(t))²)1/2, wherein L* (0) is the brightness value at time 0 before the self-tanning composition has been applied to the substrate and L*(t) is the brightness value at a time (t) after application of the composition, and similar values for a* and b* as a function of time. The rate of tanning, as gauged by ΔE as a function of time, was found to increase more rapidly for systems that included the potentiator in comparison to the control formulation, and in other cases, the final skin color also was darker as gauged by the ΔE values.

The impact of adding a potentiator to a self-tanning composition on the color of the composition also was measured using a color meter. In comparison to the same amount of amodimethicone or lysine directly to the composition, composition containing the potentiator loaded microspheres exhibited a significant improvement in the color using either the ΔE or the Δb* index, such that in some cases, the self-tanning composition had only a slight yellow color.

As demonstrated below, the present compositions are color stable because the potentiator is loaded onto the polymeric microspheres. In particular, the present compositions, when compared to an identical composition absent the loaded microspheres, has a ΔE of about 20 or less.

An in vivo determination of self-tanning was performed by blocking out a defined area of skin, measuring skin color in that area with a color meter, and then applying a measured quantity of the test formulation to the defined area. The color meter then was used to measure skin color as a function of time after application of the test formulation.

### Examples

1) Loading of glycine: A glycine solution was prepared by adding 7.01 g glycine to 95.00 g DI (deionized) water. The resulting mixture was stirred until the glycine had completely dissolved. The resulting glycine solution (29.88 g) was added to 9.96 g POLY-PORE® E200 microparticles in several portions with care being taken to make sure that the POLY-PORE® E200 was uniformly wetted. POLY-PORE® E200 is microspheres of a copolymer allyl methacrylate and ethylene glycol dimethacrylate copolymer. The glycine-loaded microparticles were placed in a vacuum oven at 50° C. overnight to remove water. The resulting material was a free-flowing, white powder that contained 16.9%, by weight, glycine.

2) Loading of lysine: A lysine solution was prepared by adding 5 g of lysine to 95.00 g DI water. The resulting mixture was stirred until the lysine had completely dissolved. The resulting lysine solution (15 g) was added to POLY-PORE® E200 microspheres (3.00 g) and, after mixing until homogeneous, the lysine-loaded microparticles were placed in a 50° C. vacuum oven overnight to provide a free-flowing powder containing of 13.0%, by weight, lysine.

3) Loading of GE SF1708: GE SF 1708 (a silicone polymer containing pendant amino groups, INCI name: amodimethicone) was loaded onto POLY-PORE® E200 microspheres either by a direct addition of the viscous silicone fluid or by first dissolving the silicone fluid in a suitable solvent, such as hexanes. As an example of a direct addition, 15.00 g of GE SF 1708 was added stepwise, and with an appropriate amount of mixing, to 5.00 g of POLY-PORE® E200 to give a final material that contained a 75%, by weight, load of GE SF 1708. Similar loaded microparticles were prepared wherein the level of GE SF 1708 varied from 25% to 75%, by weight. To prepare loaded microparticles of GE SF 1708 from a hexanes solution, first 6.24 g of GE SF 1708 was added to 12.48 g of hexanes and stirred until a clear solution formed. The solution was added to 18.73 g of POLY-PORE® E200 in a stepwise process and with sufficient stirring to ensure a homogeneous mixture, then the loaded particles were dried in a vacuum oven at 50® C. overnight. The resulting material was a free-flowing pouch containing a 25% entrapment of GE SF 1708 in POLY-PORE® E200.

4) Loading of Dow Corning DC 2-2856 (INCI name: amodimethicone): To prepare this material, 11.42 g of a 50% solution of DC 2-2856 in hexanes was added to 5.71 g of POLY-PORE® E200 microparticles. The resulting microparticles then were dried in a vacuum oven overnight to provide loaded microparticles containing 50%, by weight, of DC 2-2856.

5) Preparation of a DHA Containing Self-Tanning Composition. To test the ability of a potentiator loaded onto microparticles to enhance the rate of tanning or to minimize adverse aesthetics on the formulation, the potentiator loaded microparticles were added to an oil-in-water (o/w) composition that contained DHA. In this test, commercial compositions were used, such as those sold by Neutrogena (Sunless Tanning Lotion, Deep Glow), Avon Sun (Self-tanning Lotion, Medium/Dark), and Walgreen's Paradise Gold (Deep Dark Tan). The loaded microspheres were added to the commercial composition with sufficient agitation to ensure a homogeneous composition.

6) Loading of amodimethicone on POLYTRAP 6603. Amodimethicone (GE SF 1708) (33.33 g) was added to 16.7 g hexanes, and the mixture was stirred until a homogeneous solution formed. This solution then was added to 100 g of POLYTRAP 6603 in a stepwise process to ensure that a homogeneous loading was achieved. POLYTRAP microparticles are a copolymer of ethylene glycol dimethacrylate and lauryl methacrylate. The resulting amodimethicone-loaded microparticles then were dried in a vacuum oven overnight at 45° C. to give free-flowing particles that contained 25%, by weight, amodimethicone.

7) Loading of amodimethicone in POLYTRAP 6603: Amodimethicone was dissolved in 50 g hexanes, and this solution was added to 100 g of POLYTRAP 6603 microparticles in a stepwise process to provide a homogeneous distribution of the amodimethicone. The amodimethicone-loaded microparticles then were dried in a vacuum oven overnight at 40° C. to give free-flowing particles that contained 50%, by weight, amodimethicone.

8) To 40 g of the amodimethicone-loaded microparticles of Example 6, was added 40 g of Shea butter (melted at 80° C.) with mixing, and in a stepwise addition to allow for uniform incorporation of the wax. The microparticles contained 12.5%, by weight, amodimethicone and 50%, by weight, Shea butter.

9) Example 8 was repeated, except stearyl alcohol (melted at 80° C.).was used instead of Shea butter to give microparticles containing POLYTRAP 6603 37.5%, amodimethicone 12.5%, and stearyl alcohol 50%, by weight.

10) To 40 g, of the amodimethicone-loaded microparticles of Example 6 was added 80 g of Shea butter (melted at 80° C.) to give microparticles containing POLYTRAP 6603 24.75%, amodimethicone 8.25%, and Shea butter 67%, by weight. Similar microparticles were prepared wherein the Shea butter was replaced by stearyl alcohol.

11) To 50 g of the amodimethicone-loaded microparticles of Example 7 was added 100 g of stearyl alcohol (melted at 80° C.) to give microparticles containing POLYTRAP 6603 25%, amodimethicone 2.5%, and stearyl alcohol 50%, by weight.

12) To 10 g of POLYTRAP 6603 microparticles loaded with 12.4%, by weight, lysine was added 20 g of Shea butter (melted at 80° C.) to give microparticles containing POLYTRAP 6603 28.9%, lysine 4.1%, and Shea Butter 67%, by weight.

13) To 50 g of the amodimethicone-loaded microparticles of Example 7 was added 100 g of molten Shea butter (melted at 80° C.) with sufficient stirring to prepare a homogeneous mixture. The microparticles contained POLYTRAP 6603 16.5%, amodimethicone 16.5%, and 67% Shea butter, by weight.

14) A homogeneous solution was prepared by mixing 50 g amodimethicone, 50 g dimethicone (10 centistoke), and 100 g hexanes. This mixture then was added to 100 g of POLYTRAP 6603 microparticles. The resulting loaded microparticles were dried in a vacuum oven overnight at 40° C.-45° C. to give microparticles containing of POLYTRAP 6603 50%, 25% amodimethicone, and 25% dimethicone, by weight.

15). To 15 g of the loaded POLY-PORE microparticles of Example 3 was added 15 g of molten Shea butter (80° C.) to give final loaded POLY-PORE microparticles containing 12.5% amodimethicone and 50% Shea butter, by weight.

16) To 15 g of the loaded POLY-PORE microparticles of Example 3 was added 30 g of molten Shea butter (80° C.) to give final loaded POLY-PORE microparticles containing 8.25% amodimethicone and 67% Shea butter, by weight.

17) To 10 g of the loaded POLY-PORE microparticles of Example 4 was added 10 g of molten stearyl heptonate to give final loaded POLY-PORE microparticles containing 25% amodimethicone and 50% stearyl heptonate, by weight.

18) A mixture of 25 g amodimethicone and 50 g cyclomethicone was stirred until homogeneous. This mixture then was added to 25 g of POLYTRAP 6603 to give a white fluffy mixture.

19) To 10 g of the loaded POLYTRAP 6603 microparticles of Example 18 was added 10 g molten stearyl heptonate to give final loaded POLYTRAP microparticles containing 12.5% amodimethicone, 25% cyclomethicone, and 50% stearyl heptonate, by weight.

20) For some experiments, an o/w formulation was used as a base into which an appropriate amount of DHA was added (from a 50 wt % solution in water) followed by the addition of POLY-PORE E200 microparticles loaded with a potentiator. The base formulation is shown below.

| | | Wt. (%) | Batch (g) |
|---|---|---|---|
| A | DI Water | 59.9 | 299.5 |
| A | Keltrol T (2%) | 15 | 75 |
| A | Na₂EDTA | 0.1 | 0.5 |
| B | Cetearyl Alcohol | 3 | 15 |
| B | Lipomulse 165 (glyceryl | 1.5 | 7.5 |
| | stearate and PEG-100 | | |
| | stearate) | | |
| B | Caprylic/Capric | 12.5 | 62.5 |
| | Triglyceride | | |
| B | Eutanol G (octyl | 2 | 10 |
| | dodecanol) | | |
| B | Brij 721 (POE (21) | 2.5 | 12.5 |
| | stearyl ether) | | |
| B | Behenyl Alcohol | 2.5 | 12.5 |
| C | Phenonip (preservative) | 1 | 5 |
| | Total | 100 | 500 |

Premix ingredients A
Premix ingredients B
Heat A to 75° C.
Heat B to 75° C.
Add phase B to A and homogenize
Cool to 40° C.
Add phase C

21) To the cosmetic base described in Example 15 was added either 2 wt. % POLY-PORE E200 loaded with 25% amodimethicone, or 4 wt. % POLY-PORE E200 25 wt. % amodimethicone. For all formulations, the DHA content was adjusted to 5%, by weight. The tanning ability was measured by the in vitro method of R. Jermann et al. described above, and the results are summarized in the following table.

| | | Delta E, 2% POLY- | Delta E, 4% POLY- |
|---|---|---|---|
| Time | Delta E, | PORE E200/25% | PORE E200/25% |
| (hrs) | Control | Amodimethicone | Amodimethicone |
| 1 | 1.24 | 4.34 | 6.92 |
| 3 | 1.52 | 4.54 | 6.76 |
| 6 | 6.46 | 9.40 | 11.9 |
| 22 | 20.0 | 20.9 | 22.9 |

22) To the cosmetic base described in Example. 15 was added 4 wt. % of POLY-PORE E200 microparticles loaded with 75%, by weight, of a 1:2 mixture of amodimethicone and cyclomethicone. For all formulations, the DHA content was adjusted to 5 wt %.

| | | Delta E, 4% POLY-PORE E200/75% |
|---|---|---|
| Time | Delta E, | (33% amodimethicone/66% |
| (Hrs) | Control | cyclomethicone) |
| 1 | 1.2 | 5.64 |
| 3 | 1.5 | 6.91 |
| 6 | 6.46 | 10.1 |
| 22 | 20.1 | 19.5 |

23) To the cosmetic base describe in Example 15 was added 4 wt % of POLY-PORE E200 loaded with 50% lysine, and to a second emulsion, 4 wt % of POLY-PORE E200 loaded with 50% arginine. For all the formulations, the DHA content was adjusted to 5 wt % in the final formulation.

| | | Delta E, POLY- | Delta E, POLY- |
|---|---|---|---|
| Time | Delta E, | PORE E200/50% | PORE E200/50% |
| (Hrs) | Control | lysine | arginine |
| 1 | 0.33 | 3.50 | 0.35 |
| 3 | 3.40 | 11.6 | 3.21 |
| 6 | 8.17 | 17.9 | 8.57 |
| 22 | 21.4 | 32.0 | 21.8 |

24) For the following experiments, an o/w formulation was used as a base into which an appropriate amount of DHA was added (from a 50 wt % solution in water), followed by the addition of POLY-PORE E200 microparticles loaded with a potentiator. The base formulation is shown below.

| | | Wt. (%) | Batch (g) |
|---|---|---|---|
| A | DI Water | 60.3 | 301.7 |
| A | Keltrol T (2%) | 8 | 40 |
| A | Glycerin (96%) | 3 | 15 |
| A | Magnesium Aluminum Silicate | 2 | 10 |
| B | Cetearyl Alcohol | 3 | 15 |
| B | Glyceryl stearate and PEG-100 | 3 | 15 |
| | stearate | | |
| B | Caprylic/Capric Triglyceride | 9 | 45 |
| B | Dimethicone (100 cst) | 2 | 10 |
| B | Ceteryl alcohol/cetearth 20 | 2 | 10 |
| B | Myristyl Myristate | 0.5 | 2.5 |
| C | Citric acid (20% solution) QS | 1 | 5 |
| | to pH 3.8 | | |
| C | Germaban II (Sutton) | 1 | 5 |
| | Total | 100 | 500 |

Premix ingredients A
Premix ingredients B
Heat A to 75° C.
Heat B to 75° C.
Add phase B to A and homogenize
Cool to 40° C.
Add phase C

To make the formulated products discussed in the examples, an appropriate amount of 50% DHA aqueous solution was added to the emulsion together with the appropriate quantity of loaded microparticles.

25) The impact on the color of the self-tanning composition after the addition of the potentiator can be assessed by measuring the color of the formulation with a color meter. The same protocol was used to measure the color of the samples as was described in Example 16 above. The measurements were either made with a Hunter color meter (Labscan 2) or an X-Rite SP62. Both instruments gave comparable results. In all cases, the potentiator, either free or loaded onto microspheres, was added to the composition to give a total concentration of the potentiator of 1 wt %. All of the compositions also contained 5 wt % DRA. The color of the composition was measured within 24 to 36 hours after adding the potentiator to the composition. The LE and Δb* values are calculated with respect to the control formulation.

| Sample (% by weight) | L* | a* | b* | ΔE | Δb* |
|---|---|---|---|---|---|
| Control (5% DHA) | 93.6 | −0.59 | 0.31 | | |
| 1% amodimethicone | 86.3 | 0.61 | 27.2 | 28.5 | 27.6 |
| 12.1% POLYTRAP 6603/8.3%, | 92.6 | −1.6 | 5.0 | 5.5 | 5.3 |
| amodimethicone 67%, | | | | | |
| Shea butter | | | | | |
| 6.1% POLYTRAP 6603/16.5%, | 91.4 | −1.3 | 4.1 | 4.5 | 3.9 |
| amodimethicone 67%, | | | | | |
| Shea butter | | | | | |
| 2% POLYTRAP 6603/50% | 89.7 | 0.68 | 16.3 | 16.6 | 16.2 |
| amodimethicone | | | | | |
| 4% POLYTRAP 6603/25% | 94.5 | −1.4 | 13.8 | 13.7 | 13.7 |
| amodimethicone | | | | | |
| 0.1% Lysine | 87.7 | 0.59 | 13.4 | 14.9 | 13.6 |
| 2.4% POLYTRAP 6602/4.1% | 90.2 | −0.44 | 11.6 | 7.0 | 11.9 |
| lysine 67% Shea butter | | | | | |

26) Using the formulation base described in Example 19, a control formulation that contained 5% DHA, by weight, was prepared. A second formulation containing 5% DHA plus 12.1% of loaded POLYTRAP 6603 microparticles containing 8.3% amodimethicone and 67% Shea butter, by weight, also was prepared. The bicep area of a human panelist was marked into two 9 cm² areas. The color of the skin was measured using X-Rite SP 62 color meter. To one area was applied 45 mg of the control formulation and to the second area was applied the formulation containing the loaded POLYTRAP 6603 microparticles. The color of the skin was measured as a function of time.

Between the end of the first day and the 22-hour time point, the panelist washed as normal. The results are tabulated with respect to the color change (Delta E) from the skin before application of the lotions.

| | | Delta E POLYTRAP |
|---|---|---|
| Time (Hrs) | Delta E Control | Formulation |
| 1 | 1.0 | 1.2 |
| 2.25 | 1.7 | 1.9 |
| 3 | 2.7 | 3.0 |
| 4 | 3.5 | 3.7 |
| 6 | 4.2 | 5.1 |
| 9 | 5.0 | 5.2 |
| 22 | 4.2 | 5.6 |

Obviously, many modifications and variations of the invention as hereinbefore set forth can be made without departing from the spirit and scope thereof and, therefore, only such limitations should be imposed as are indicated by the appended claims.

1. A self-tanning composition comprising
(a) a self-tanning compound; and
(b) an amine potentiator loaded on polymeric microparticles.

2. The self-tanning composition of claim 1 comprising about 0.1% to about 10%, by weight, of the self-tanning compound.

3. The self-tanning composition of claim 1 wherein the self-tanning compound comprises dihydroxyacetone.

4. The self-tanning composition of claim 1 wherein the self-tanning compound comprises L-erythrulose.

5. The self-tanning composition of claim 1 comprising about 0.1% to about 10%, by weight, of the amine potentiator.

6. The self-tanning composition of claim 1 wherein the amine potentiator comprises an amino acid.

7. The self-tanning composition of claim 6 wherein the amino acid comprises lysine, glycine, arginine, or a mixture thereof.

8. The self-tanning composition of claim 1 wherein the amine potentiator comprises a diamine, a triamine, or a mixture thereof.

9. The self-tanning composition of claim 8 wherein the diamine or triamine comprises 1,2-ethanediamine, 1,3-propanediamine, 1,4-butanediamine, 1,6-hexanethylenediamine, diethylenetriamine, triethylenetetraamine, N,N′-dimethylethylenediamine, N,N′-diethylethylenediamine, N,N′-diisopropylethylenediamine, N,N′-di-n-propylethylenediamine, N,N′-di-n-butylethylenediamine, N,N′-di-n-hexylethylenediamine, N,N′-dibenzylethylenediamine, N,N′-di-(2-carboxyethyl)-ethylenediamine, N,N′-di-(2-hydroxyethyl)-ethylenediamine, N-ethylethylenediamine, N-n-propylethylenediamine, N-isopropylethylenediamine, N-n-butylethylenediamine, N-sec-butylethylenediamine, N-hexylethylenediamine, N-phenylethylenediamine, N-benzylethylenediamine, N.-(2-hydroxyethyl),-ethylenediamine, N-(3-hydroxypropyl)-ethylenediamine, N-[3-(trihydroxysilyl)-propyl]-ethylenediamine, N-[(3-(trimethoxysilyl)-propyl]-ethylenediamine, and N-naphthylethylenediamine, or mixtures thereof.

10. The self-tanning composition of claim 1 wherein the amine potentiator comprises an amino-containing polymer.

11. The self-tanning composition of claim 10 wherein the amino-containing polymer comprises amodimethicone, methoxy amodimethicone/silesquioxane copolymer, a linear polyethylenediamine, a branched polyethylenediamine, a polyethylenimine, a dendritic amino polymer, poly(lysine), poly(argine), or mixtures thereof.

12. The self-tanning composition of claim 1 wherein the polymeric microparticles are selected from the group consisting of a copolymer, of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

13. The self-tanning composition of claim 1 wherein the polymeric microparticles comprise a copolymer of allyl methacrylate and ethylene glycol dimethacrylate, a copolymer of ethylene glycol, dimethacrylate and lauryl methacrylate, or a mixture thereof.

14. The self-tanning composition of claim 1 wherein the amine potentiator is loaded onto the polymeric microparticles in an amount to provide loaded microspheres containing about 2% to about 80%, by weight, of the potentiator.

15. The self-tanning composition of claim 1 further comprising a compound capable of modifying a rate of release of the amino potentiator from the polymeric microparticles or the reactivity of the amine potentiator.

16. The self-tanning composition of claim 15 wherein the modifying compound is selected from the group consisting of a C₈-C₂₀ alcohol, a fatty alcohol ethoxylated with one to three moles of ethylene oxide, a C₈-C₂₀ fatty acid, a hydrocarbon, a wax, a fat, an oil, an ester containing at least 10 carbon atoms, and mixtures thereof.

17. The self-tanning composition of claim 1 wherein the amino potentiator is adsorbed on polymeric microparticles.

18. The self-tanning composition of claim 1 wherein the amino potentiator is absorbed on polymeric microparticles.

19. The self-tanning composition of claim 1 wherein the amino potentiator is entrapped in polymeric microparticles.

20. The self-tanning composition of claim 1 wherein the composition is in a form of a water-in-oil emulsion, an oil-in-water emulsion, an anhydrous stick, or an aqueous gel.

21. The self-tanning composition of claim 1 wherein the composition is color stable compared to an identical composition free of the amine potentiator loaded on polymeric microparticles.

22. A method of tanning the skin comprising applying a composition of claim 1 to the skin.
